(19) United States
(12) Patent Application Publication
(10) Pub. No.: US 2011/0081684 A1
(43) Pub. Date: Apr. 7, 2011

(54) METHOD FOR CARRYING OUT AN ENZYMATIC REACTION

(76) Inventors: Christoph Gauer, Munich (DE); Wolfgang Mann, Neudrossenfeld (DE); Marianna Alunni-Fabbroni, Munich (DE)

(21) Appl. No.: 12/312,906

(22) PCT Filed: Sep. 18, 2007
(86) PCT No.: PCT/EP2007/008123

§ 371 Date: Nov. 2, 2009

(51) Int. Cl. C12P 19/34; C12M 1/34

(52) U.S. Cl. 435/91.1; 435/288.7

(57) ABSTRACT

The invention relates to a method for carrying out an enzymatic reaction, in particular for carrying out a polymerase chain reaction (PCR). Said method consists of the following steps: at least one eukaryotic cell is removed from a starting material; the cell core or cores of the eukaryotic cell(s) is/are coloured; at least one eukaryotic cell is deposited on a reaction point of a solid substrate in a liquid volume of less than 10 μl; it is detected whether at least one coloured cell core is present on a reaction point of the substrate, subsequently, an enzyme and optionally a reaction buffer is added to the eukaryotic cell(s) and the enzymatic reaction is subsequently started. Preferably, the claimed invention is carried out using a flow cytometer.

The present invention relates to a method for carrying out an enzymatic reaction, in particular for carrying out a single cell polymerase chain reaction and also to a substrate on which one or more eukaryotic cells are provided.

As a result of the sensitivity of enzymes with respect to contaminants such as salt, organic solvents and the like cleaned samples must be used in enzymatic reactions in order to ensure an effective course of the enzymatic reaction. This applies in particular also for enzymatic reactions in which the substrate of the enzyme is a nucleic acid, for example DNA. Examples for such enzymatic reactions are restriction hydrolyses, ligations and amplifications reactions, such as for example the polymerase chain reaction (PCR).

A series of methods are known in order to prepare DNA with adequate purity for an enzymatic reaction. Moreover, a number of corresponding kits are commercially offered for the purification of nucleic acids in particular DNA, such as for example QIAamp® DNA Blood Kit for the purification of genomic DNA from blood. The object of these methods and kits is to be able to separate from the DNA cell components such as lipids, proteins—for example DNA nuclease and the like—which would disturb a later enzymatic reaction. The purity of nucleic acid solutions is regularly expressed by the quotient of the absorption at 260 nm divided by the absorption at 280 nm and this quotient should be greater than 1.8 for the use of the nucleic acid solutions in enzymatic reactions. Further enzymatic reaction inhibiting contaminants in addition to proteins, salts and organic solvents are for example the hem groups of the hemoglobin of human erythrocytes, the central porphyrin scaffold of which is suitable to complex enzymatic cofactors such as Mg²⁺ and thus to prevent the enzymatic reaction. As a result of the hemoglobin a PCR or similar amplification reactions are not possible from full blood.

For the named reasons the classical molecular biological diagnostics and/or human genetics are nowadays split up into the areas of sample preparation namely the regular extraction of the nucleic acids from cells, the carrying out of the enzymatic reaction, for example amplification such as PCR, and also detection, which for example takes place via fluorescence. Seen economically, the sample preparation mainly represents the largest cost factor of the total process because the other steps, namely the carrying out of the enzymatic reaction and also the subsequent detection can be better miniaturized than the sample preparation, whereby the costs for the above-named steps can be reduced.

Recently methods for carrying out enzymatic reactions have been proposed in which individual cells are used as an enzyme substrate instead of isolated and purified nucleic acid. This has the advantage that a costly sample preparation can be dispensed with. Because an individual cell contains the whole genome of an organism a few cells, or theoretically even a single cell, are sufficient in order to carry out an enzymatic reaction. This method, however, only leads to positive results in about 50% of the cases, i.e. in approximately the half of all cases the enzymatic reaction does not take place. The reason for this in many of the cases is that significant quantities of contaminants frequently remain in the sample during the isolation of the individual cell which inhibit the subsequent enzyme reaction.

False negative results are a further problem which occurs in practice with enzymatic reactions carried out on individual cells. In an enzymatic reaction carried out on individual cells, a check must be made prior to carrying out the reaction whether a cell is present in the reaction vessel in a form susceptible to the enzyme reaction in order, in the case in which no reaction product, for example no PCR product, is obtained in the enzyme reaction, for example in a PCR, to be able to conclude unambiguously that no binding sites for the primers used in the PCR are present in the DNA of the cell that is presented. If this check is not made then the failure of the PCR can also be caused by no cell having been deposited in the reaction vessel as a result of preparation errors. However, in the known methods, incorrect results frequently occur even if a check is made prior to carrying out the enzymatic reaction whether a cell is present in the reaction vessel. This check is frequently so integrated into the method that the cell is first marked for example with a fluorescence-marked antibody for the surface protein of the cell membrane and sorted with a FACS flow cytometer and deposited onto a glass carrier or a similar substrate before the glass carrier is investigated with a microscope with respect to the presence of a cell, in order to subsequently carry out the enzymatic reaction of the addition after the required buffer and of the enzyme.

However, in this way of proceeding, a situation frequently arises, as one can find by control samples, that one detects fluorescence under the microscope but a subsequent PCR takes place negatively although the primers used in the PCR are definitively compatible with the nucleic acid contained in the cell, i.e. the nucleic acid has binding sites for the primers used in the PCR. The cause for this false result are in this case fluorescence artifacts which are for example formed as a result of the agglomeration of antibodies, without a cell having being deposited on the substrate. The user would in this case achieve the wrong result, that the cell does not contain DNA compatible with the primers that are used, although the missing PCR product can simply be attributed to the fact that no cell was deposited on the substrate.

In the above-named manner of proceeding, no fluorescence is also frequently detected under the microscope (from which it could be incorrectly concluded that no cell was deposited on the substrate) although PCR products are obtained in a subsequent PCR. This wrong result is in the most frequent cases to be attributed to the fact that the cell was mechanically destroyed on meeting the substrate and could no longer be recognized under the microscope, although the DNA of the now destroyed cell that is used was present on the substrate in a form accessible to the subsequent PCR.

The object of the present invention is thus to make available a method for the carrying out of an enzymatic reaction in which the extraction of nucleic acids from cells can be dispensed with, which is simple and quick to carry out and which in particular also leads to an unambiguous result when using a few cells, in particular one cell.

In accordance with the invention this object is satisfied by a method in accordance with patent claim 1 and in particular by a method for carrying out an enzymatic reaction, in particular a PCR, with a sample containing at least one eukaryotic cell, including the following steps:

a) making available a starting material containing at least one eukaryotic cell,

b) removal of at least one eukaryotic cell from the starting material,

c) coloring the cell nucleus or the cell nuclei of the eukaryotic cell(s),

d) depositing at least one eukaryotic cell on a reaction site of a solid substrate in a liquid volume of less than 10 μl,

e) detecting whether a colored cell nucleus is present on a reaction site of the substrate and also

f) carrying out an enzymatic reaction with the at least one eukaryotic cell on the substrate (11),

wherein the method step e) is carried out after the method steps c) and d).

Since, in the method of the invention, the cell nucleus of the eukaryotic cell(s) to be deposited on a reaction site of the substrate is first colored and the substrate is investigated in the method step e) for cell nucleus coloring, or for the presence of at least one colored cell nucleus, it can be unambiguously determined whether DNA is present in a form accessible for the enzyme used in the enzymatic reaction prior to carrying out the enzymatic reaction and indeed independently of whether the cell was mechanically lysed during deposition on the substrate or not. In the same way false results as a result of some form of fluorescence artifacts can also be precluded by this method step. Consequently, the above described cases of false positive results occurring with the methods known from the prior art are reliably precluded by the method of the invention so that unambiguous results are obtained with the method of the invention. Moreover, very time-consuming and cost-intensive extraction of nucleic acid from cells can be dispensed with in the method of the invention in that one or more eukaryotic cells are used which have the entire genetic material of an organism.

In accordance with the invention the method steps b), c) and d) can be carried out in any desired sequence. In particular the nucleus coloring in accordance with the method step c) can be carried out prior to depositing the eukaryotic cell(s) on a reaction site of the substrate in accordance with method step d) and in particular also prior to or after the removal of at least one eukaryotic cell from the starting material in accordance with the method step b).

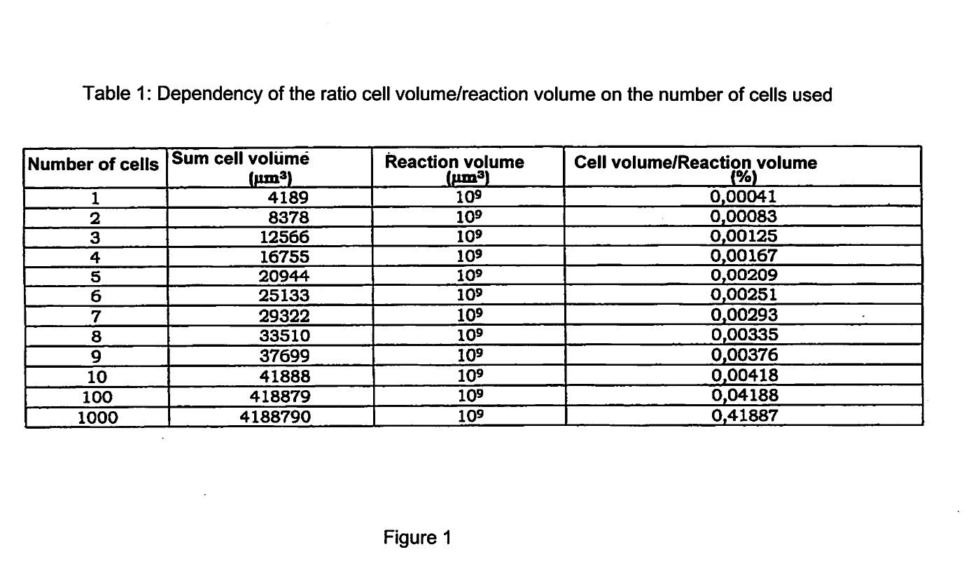

The method in accordance with the invention is particularly suitable for carrying out an enzymatic reaction, in particular a PCR on individual eukaryotic cells or a few eukaryotic cells. Preferably, in the method step d) a maximum of 10 eukaryotic cells, preferably between 1 and 5 cells, particularly preferably between 1 and 3 cells and especially preferably 1 or 2 cells are deposited per reaction site of the substrate. In particular the carrying out of the single cell reaction is preferred in which case precisely one cell is deposited on a reaction site of the substrate. By the use of a few eukaryotic cells in the enzymatic reaction it is ensured that only minimal quantities of the contaminants present in the intracellular fluid reach the substrate on which later the enzymatic reaction takes place. This will be made clear with respect to the following computational example. Human cells or basically mammal cells have size differences. Erythrocytes for example have an average cell diameter of 7.5 μm whereas granulocytes have a diameter between 9 and 16 pm and the cell diameter for lymphocytes amounts, depending on the organism, to 5 to 18 μm. Bacterial cells in contrast have an average cell diameter of between 1 and 5 μm. Starting from an average cell diameter of 10 μm the volume of a cell amounts, on assuming a of its spherical form of the cell, to 4/3·π·r³, i.e. to ca. 4200 μm³. Accordingly, if a cell is used in a PCR with a standard reaction volume of 1 μl corresponding to 10⁹ μm³ the ratio of the cell volume to the total reaction volume amounts to approximately 0.00042%. If in contrast 10, 100 or even 1,000 cells are used in the same reaction volume then the above-named ratio of the cell volume to the total reaction volume increases to 0.0042% for 10 cells, to 0.042% for 100 cells and to 0.42% for 1,000 cells. This association is shown in FIG. 1. Since cells, in addition to the nucleic acids which are the later substrate for the enzymatic reaction, mainly consist of contaminants such as proteins, lipids and the like which potentially disturb the enzymatic reaction, the proportion of the contaminants present intracellularly in the cells which are introduced into the enzymatic reaction is drastically lowered by the reduction of the quotient of the cell volume to the reaction volume.

The removal of the individual eukaryotic cells from the starting material in accordance with the method step b) can take place with any method known to the person skilled in the art for this purpose. By way of example the individual eukaryotic cells can be removed with a glass capillary from a cell suspension, which is optionally diluted prior to the extraction to a suitable value and which can contain exclusively eukaryotic cells or a mixture of eukaryotic cells and prokaryotic cells. For example the mmi Cellector® of the company MMI Molecular Machines & Industries AG for the micromechanical removal of individual eukaryotic cell(s) from the starting material in accordance with the method step b) with a capillary has proved to be particularly suitable.

In order to be able to check or control the number of the eukaryotic cells deposited on the reaction site of the substrate, the absolute number of the eukaryotic cell(s) to be deposited or deposited per reaction site is determined prior to or during the deposition of the at least one eukaryotic cell on the reaction site of the substrate. This can for example take place with a microscope, and a quantification of the absolute number of the eukaryotic cell(s) deposited on a reaction site of the substrate, in particular a quantification by an optical microscope or by fluorescence microscope, has proved to be particularly suitable.

The method of the present invention is also in particular not restricted with respect to the nature of the cell nucleus coloring or of the dye that is used in the method step c). Good results are in particular obtained with dyes which are specific for the cell nucleus, i.e. do not color other cell structures in addition to the cell nucleus or only to a subordinate degree. Examples for suitable dyes are those which are selected from the group consisting of hematoxyline, alum carmin, alcoholic boraxamine solution, paracarmin, naphthazarin, carmin acetic acid and desired combinations hereof. Fluorescent dyes have in particular also proved successful for this purpose, preferably those selected from the group consisting of 7-amino-actinomycine D (7-AAD), acridine orange, BOBO-1, BOBO-3, DAPI Nucleic Acid Stain, dihydroethidium, ethidium bromide, ethidium homodimer-1, hexidium iodide, Hoechst 33258, Hoechst 33342, Hoechst 34580, LDS 751, Nissl substance, nuclear yellow, propidium iodide, SYTO 11, SYTO 13, SYTO 16, SYTOX green stain, SYTOX orange, TO-PRO-3, TOTO-3, YO-PRO-1, YOYO-1 and desired combinations hereof.

As a further development of the concept underlying the invention it is proposed to deposit the at least one eukaryotic cell in the method step d) on an inner hydrophilic region of a reaction site of the substrate which is surrounded by a hydrophobic region. Through the deposition of at least one eukaryotic cell onto a hydrophilic region of a reaction site of the substrate surrounded by a hydrophobic region the formation of a liquid drop formed from the liquid adhering to the at least one eukaryotic cell or from the liquid supplied to the at least one eukaryotic cell after the deposition on the reaction site is made possible, which adheres comparatively strongly to the substrate, so that the subsequent enzymatic reaction can be carried out directly on the reaction site without the eukaryotic cell having to be transferred into a closed reaction vessel or the like. In this way work-intensive and time-consuming transfer steps are avoided. Furthermore, it makes it possible for a plurality of samples to be prepared in parallel on the substrate comprising a corresponding number of hydrophilic reaction sites, spatially separated from one another, without the danger existing that the liquid drops which lie spatially close to one another, mix with one another with small vibrations or as a result of running of liquid drops as a consequence of a drop volume which is too large.

Moreover it has proved to be advantageous when the inner hydrophilic region of the reaction site(s) on the substrate are made substantially circular and is or are surrounded, preferably concentrically, by a hydrophobic region which is also substantially of circular ring-shape.

An even better formation of the liquid drops on the substrate is achieved when the hydrophobic region of the substrate surrounding the inner hydrophilic region of the reaction site is surrounded on the substrate at the outer side of the substrate by at least one middle hydrophilic region which is preferably of substantially circular ring-shape and which surrounds the hydrophobic region in particularly preferably concentrically. The middle hydrophilic circular ring is preferably surrounded at the outer side by an outer hydrophobic region. Thus a particularly preferred arrangement consists of a circular hydrophilic region which is concentrically surrounded by two circular rings, with the inner of the two circular rings being hydrophobic and the outer of the two circular rings being hydrophilic and with the outer hydrophilic circular ring being surrounded at the outer side by a hydrophobic region.

Particularly good results are in particular obtained when the hydrophilic property of the inner hydrophilic region of the reaction site and the hydrophobic properties of the region surrounding it are set such that, when a few 10 μl's of water are applied onto the reaction site, a water drop is formed with a contact angle between 20° to 70°, preferably between 30° and 60° and particularly preferably from 40° to 50°. In this way it is ensured that a stable liquid drop forms which sticks firmly to the reaction site so that the liquid drop does not separate from the glass plate or run on the glass plate with even the smallest vibrations of the substrate such as occur during the transport of the substrate, for example in a laboratory.

The diameter of the inner hydrophilic region of the reaction site preferably amounts to between 0.3 and 3 mm providing it is of substantially circular shape as is preferred.

In order to enable the preparation in parallel of a plurality of samples it is proposed, as a further development of the concept of the invention, to provide from 2 to 1,000, preferably from 12 to 256, particularly preferably from 24 to 96 and quite especially preferably 48 different reaction sites on the substrate each including a substantially circular inner hydrophilic region, with the inner hydrophilic regions respectively being concentrically surrounded by a substantially circular ring-like hydrophobic region which is surrounded at the outer side by a middle hydrophilic region of substantially circular ring-shape with an outer hydrophobic region preferably again following the middle hydrophilic region at the outer side.

The method of the invention is not limited with respect to the nature of the substrate that is used. For example the substrate can be a reaction vessel of plastic it is likewise just as well possible to use a micro-titer plate as a substrate, for example a 96-well, 128-well, 256-well or 528-well micro titer plate. As an alternative to this it has proved to be advantageous to provide an object carrier as the substrate, particularly preferably an object carrier whose surface is coated with epoxy and is subdivided by lithographically manufactured hydrophilic and hydrophobic regions into individual reaction sites or anchor locations. Such object carriers are for example commercially sold by the company Advalytix under the trade name AmpliGrid™. An object carrier or a micro titer plate is preferably used as a substrate, with an AmpliGrid™ which includes the above described reaction sites with circular hydrophilic and hydrophobic regions being in particularly suitable as a substrate.

In order to minimize the quantity of any contaminants originating from the sample preparation on the carrier which could disturb the subsequent enzymatic reaction, the at least one eukaryotic cell is preferably deposited on a reaction site of the substrate in the method step d) in a liquid volume of less than 5 μl, particularly preferably of less than 2 μl and especially preferably of less than 1 μl.

For the same reason it is in particular preferred to deposit the at least one eukaryotic cell in the method step d) in a liquid volume of less than 100 nl, preferably of less than 10 n1 and particularly of maximum 1 nl on a reaction site of the substrate. This embodiment ensures that the cells used in the enzymatic reaction contain adequately little contaminants which inhibit the enzymatic reaction. This is a further particular advantage with respect to the individual cell methods known from the prior art in which the isolation of individual cells normally takes place by extracting an aliquot of a suspension of cells in cell medium, with the aliquot being deposited onto a substrate on which or in which the later enzymatic reaction is to take place. In order to carry out the enzymatic reaction enzyme as well as reaction buffer must be added to the aliquot of the suspension in order to set the ideal salt conditions and the ideal pH value for the enzymatic reaction. In order to keep the reaction volume of the enzymatic reaction as small as possible the cell suspension deposited on the substrate is concentrated in some methods by vaporization in order to vaporize the liquids surrounding the cells prior to the addition of the enzyme and of the reaction buffer. In this connection, it is however only the liquid surrounding the cells which vaporizes whereas contaminants contained in the liquid such as salts present in the liquid phase of the suspension or any proteases, lipids, nucleases, and the like remain on the substrate. These contaminants can disturb the later enzymatic reaction. This problem is solved in a simple manner in the above-named embodiment of the present invention since the cells are deposited onto a reaction site of the substrate largely without additional or extra-cellular liquid, so that the enzymatic reaction can be carried out in a minimal reaction volume with the excess extra-cellular liquid that is present having to be removed, for example by vaporization. Since the cells have no extra-cellular liquid, or only a minimal quantity of extra-cellular liquid, the number of contaminants present in the later reaction volume is restricted to a minimum, namely to the quantity of the contaminants present in the cells. Moreover, it is possible to dispense with prior time-consuming washing steps, since the cells can already be deposited in pure form onto the substrate as a result of the likewise minimal quantity of extracellular liquid. On the whole, a simple cost-favorable and rapid method results for carrying out an enzymatic reaction in which it is ensured that the enzymatic reaction takes place efficiently.

In order to avoid any decomposition of the cell components, in particular nucleic acids serving as a substrate for the enzymatic reaction prior to the addition of the enzyme, eukaryotic cell(s) are preferably deposited onto the substrate prior to the addition of the enzymes which are not lysed. In this way it is avoided that the substrate for the later enzymatic reaction is chemically destroyed by the proteases or nucleases which are set free before the start of the enzymatic reaction.

The present invention is not restricted with respect to the nature of the enzymatic reaction. Simply by way of example enzymatic reactions such as restriction hydrolyses, ligations or familiar amplification reactions, in particular PCR (polymerase chain reaction), LCR (ligase chain reaction) or RCA (rolling-circle amplification) should be named. In a PCR the reaction mixture is repeatedly subjected to temperature cycles, with each temperature cycle consisting of a denaturing step at 94° C. for the separation of the double string DNA into single string DNA, an attachment step, normally at a temperature between 40° and 60° C. for the attachment of the PCR primer to the matrix DNA and an extension step at 72° C. in which the Taq polymerase catalyzes the incorporation of nucleotides into the primer bound to the matrix DNA. Since the cells provided on the substrate burst through the initial denaturing step at 94° C. the nucleic acid of the cells is accessible for the Taq polymerase.

For the deposition of the at least one eukaryotic cell onto the reaction site of the substrate all methods known to the person skilled in the art can in principle be used.

In accordance with a further preferred embodiment of the present invention the deposition of the at least one eukaryotic cell onto a reaction site of the substrate takes place in that a liquid suspension containing at least one eukaryotic cell is supplied through a nozzle, the liquid flow or the flow of the liquid suspension is separated at the nozzle into individual liquid drops separate from one another, with the individual liquid drops each containing a predetermined number of eukaryotic cells, all or individual liquid drops are electrically charged after separation from the nozzle and the individual liquid drops are guided by an electric field whereby one or more electrically charged liquid drops are directed onto one or more reaction sites of the substrate before enzyme and optionally likewise a reaction buffer are subsequently added to the deposited eukaryotic cells and finally the enzymatic reaction is started, for example by setting the reaction solution to a suitable temperature. Since the liquid flow containing the eukaryotic cell(s) is split up at the nozzle into individual liquid drops separate from one another it can be ensured in a simple way and means, by setting the concentration of the eukaryotic cells in a liquid suspension and by setting the size of the individual liquid drops, that a predetermined number of eukaryotic cells is contained in the individual liquid drops, for example precisely one eukaryotic cell per liquid drop. By electrically charging individual liquid drops after separation from the nozzle and subsequent guidance with an electric field, the individual liquid drops can be separated from one another so that individual liquid drops can be selectively applied to a reaction side of the substrate or an intended liquid drop containing the target cells can be applied to a reaction site of the substrate. When the liquid suspension contains genetically different cells it is for example possible to statistically arbitrarily electrically charge one liquid drop whereas the other liquid drops are not electrically charged.

If the individual drops are subsequently passed through the electric field then only the electrically charged drops are deflected and applied onto the correspondingly positioned substrate. As a result of the deflection by the electric field and particularly through the speed of the liquid drops it is ensured that the eukaryotic cell(s) largely no longer contain extra-cellular liquid when they strike the substrate. The parameters during the guidance of the liquid suspension through the nozzle are preferably so set, by the separation of the liquid drops at the nozzle and during the guidance of liquid drops by the electric field, that at least one eukaryotic cell is deposited on a reaction site of the substrate in a volume of less than 100 nl, preferably of less than 10 nl and particularly preferably of a maximum of 1 nl.

The eukaryotic cell(s) are preferably hydrodynamically fed through the nozzle. This can for example take place in such a way that the liquid suspension is guided through a cannula and emerges from this through a circular opening and, after emerging from the cannula, is focused by a jacket flow of a second liquid and fed through a nozzle arranged beneath the opening of the cannula.

In order to achieve a good separation of the individual liquid drops it is proposed, in a further development of the concept of the invention, that the nozzle has an internal diameter between 1 μm and 1 mm. Particularly good results are obtained when the internal diameter of the nozzle amounts to between 10 μm and 500 μm and in particular to between 50 μm and 100 μm.

For the separation of the liquid suspension at the nozzle into individual drops all methods known to the person skilled in the art for this purpose can be used. Simply by way of example the separation of the liquid suspension at the nozzle by piezoelectric modulation is named. In piezoelectric modulation a periodic pressure fluctuation is exerted onto the liquid jet flowing through the nozzle. As a result of this liquid drops with a defined and reproducible size form at the nozzle and break away from the liquid jet. Through a corresponding setting of the concentration of the eukaryotic cells in the liquid suspension, the speed of flow of the suspension and corresponding settings of the piezoelectric modulation a situation can be achieved in which each liquid drop of a defined and reproducible size contains a predetermined number of eukaryotic cells, for example precisely one eukaryotic cell. The separation of the drops from the nozzle takes place as a result of the impulse of the pressure fluctuations aided by gravity.

The method of the invention is suitable both for the depositing of a specific number of genetically like eukaryotic cells from, for example, a cell culture medium containing only genetically like cells, for example cells of a clone onto a reaction site of a substrate and also for the deposition of a specific number of genetically like eukaryotic cells from a mixture of genetically different cells on a substrate. Moreover, with the method of the invention a specific number of genetically different eukaryotic cells from a corresponding cell mixture can be deposited onto the substrate and subjected there to an enzymatic reaction.

The first named method variant can for example be realized in that only genetically like eukaryotic cells are present in the liquid suspension, the liquid flow is however split up at the nozzle into individual liquid drops such that each liquid drop contains precisely one eukaryotic cell and as many liquid drops are electrically charged as eukaryotic cells are required on the substrate. In the subsequent guidance of the liquid drops by the electric field only the electrically charged drops are deflected and brought to a correspondingly positioned substrate. The corresponding deflection of the electrically charged liquid drops can for example take place by guiding the liquid drops through a capacitor.

As an alternative to this the second named method variant can be realized in that genetically different cells are present in the liquid suspension, for example eukaryotic cells and prokaryotic cells, an individual cell or a plurality of cells is marked with a fluorescence-marked antibody or a fluorescing dye, the liquid flow is split up into individual liquid drops separate from one another at the nozzle, the liquid drops which are individually separated at the nozzle from the liquid flow are guided through a laser beam by which the fluorescence of the individual drops is measured, the liquid drops are subsequently electrically charged in dependence on the fluorescence of the cell(s) contained therein with a specific electrical charge and the individual liquid drops are so guided through an electrical field that the liquid drops are deflected onto the substrate with an electrical charge lying in a pre-selected range.

While fluorescence-marked antibodies are preferably used when the genetically different cells are cells of different organisms, the marking of individual cells with the fluorescent dye has in particular proved to be suitable when the genetically different cells originate from the same organism. The dye can in this case, for example, be associated with a DNA probe which is specific for a gene or a gene section of a specific cell type. It is equally possible to use a plurality of different fluorescence-marked antibodies or a plurality of different fluorescent dyes in order to respectively mark genetically different cells with a specific fluorescence-marked antibody or a specific fluorescent dye. Thus, through the laser two or more different cell types can be recognized, these can later be differently electrically charged and deflected onto different substrates. In the case of two different cells to be separated in the electric field the corresponding selective deflection in the corresponding electrical field can, for example, be achieved in that the liquid drops with one target type are positively charged and the liquid drops with the other target type are negatively charged. With more than two cell types the selective separation can be achieved in that the individual different cells are respectively provided with a different quantity of electrical charge, for example the cell I with an electrical charge of X C, the cell II with an electrical charge of 2·XC, the cell III with an electrical charge of 3·XC and so forth.

In accordance with a further preferred embodiment of the present invention the eukaryotic cell(s) are dispensed onto one or more reaction sites of the substrate by means of a flow cytometer. This apparatus which is also termed a fluorescence activated cell sorter (FACS) are for example commercially sold by the companies Beckton & Dickinson and Dako.

Particularly good results are obtained when a FACS-Vantage SE flow cytometer is used as the flow cytometer.

As an alternative to the above named embodiment the deposition of the at least one eukaryotic cell on the reaction site of the substrate in accordance with step d) and/or the removal of the eukaryotic cell(s) from the starting material in accordance with method step b) can also take place by laser microdissection “(laser capture microdissection; LCM)” or by laser pressure catapultation “(laser pressure catapultation (LPC)”. Suitable apparatuses for the first named technology are for example the Veritas™ microdissection instrument of the company Arcturus which is part of the company Molecular Devices or the Leica LMD6000 of the company Leica while the PALM laser capture microdissection system of the company P.A.L.M. in Wolfratshausen is an apparatus suitable for the LPC technique.

As a further development of the concept of the invention it is proposed to use at least one AmpliGrid™ as the substrate, with the at least one AmpliGrid™ being positioned in a frame which preferably has a capacity for four different AmpliGrids™. A frame of this kind can for example be designed as a hollow frame, with the individual cutouts of the hollow frame each having the shape and size of an AmpliGrid™.

In the method of the invention one or more substrates are preferably used which each have 2 to 1,000, preferably 12 to 256 and particularly preferably between 24 to 96 and, quite especially preferred, 48 different reaction sites each including an inner hydrophilic region, with the respective numbers of the eukaryotic cell(s) deposited in the method step d) per reaction site being stored during or after the method step d) on a data carrier, for example on a hard disc.

As a further development of the concept of the invention it is proposed to carry out the detection in the method step e) of the method of the invention microscopically, preferably with an optical microscope or with a fluorescence microscope.

The method of the invention is suitable for carrying out an enzymatic reaction using all known eukaryotic cell types. In particular it is suitable for enzymatic reactions of human cells, with the method of the invention having proved particularly suitable for carrying out an enzymatic reaction on erythrocytes, granulocytes, lymphocytes, thrombocytes and cancer cells.

In the following the present invention will be described purely by way of example with reference to advantageous embodiments and to the accompanying drawings.

There are shown:

FIG. 1 the dependence of the ratio of the quotient cell volume to reaction volume of the cell number that is used,

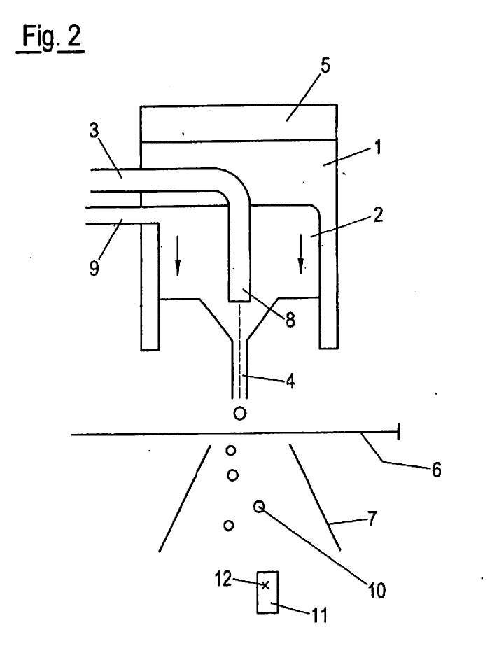

FIG. 2 an apparatus suitable for carrying out the method of the invention,

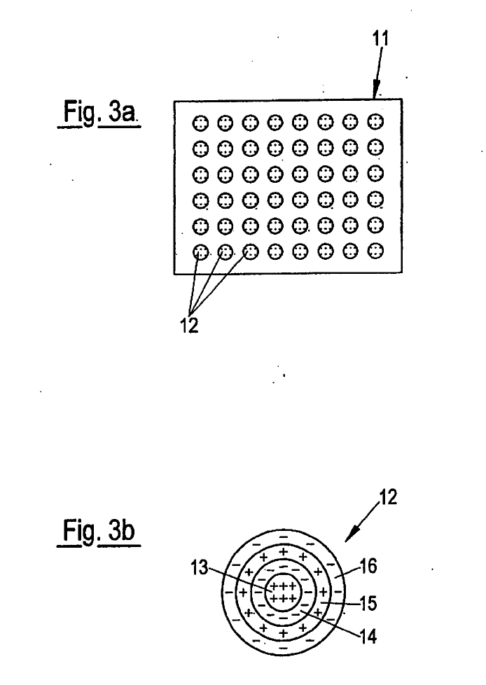

FIG. 3a a plan view of a substrate suitable for carrying out the present invention in accordance with an embodiment and

FIG. 3b a reaction site of the substrate shown in FIG. 3a.

In FIG. 1 there is shown the relationship of the quotient of the cell volume per reaction volume on the cell number that is used for cells with a diameter of 10 μm and a reaction volume of 1 μl. For a cell with a the cell diameter of 10 μm and assuming a spherical cell shape a cell volume of ca. 4200 μm³ results. Since a reaction volume of 1 μl corresponds to 10⁹ μm³, the ratio of the cell volume to the reaction volume for a cell proportioned as above in a PCR standard reaction volume of 1 μl is approximately 0.004%. If the reaction mixture contains more than one cell this ratio increases proportionally to the number of cells used. When using ten cells the corresponding ratio in the present case already amounts to 0.004% and when using 1,000 cells lead to 0.4%. Since the cells, in addition to the DNA serving as a substrate for the Taq polymerase contain further components such as proteins, lipids and the like which can inhibit the Taq polymerase, a larger ratio of cell volume to reaction volume also signifies the increasing danger that the PCR is inhibited or at least does not take place ideally. For this reason it is preferred in the method of the invention to deposit a maximum of 10 eukaryotic cells on a reaction site of the substrate. Particularly good results are obtained when a maximum of 5 eukaryotic cell and in particular a maximum of 3 eukaryotic cells are deposited on a reaction site of the substrate. The best results are obtained when precisely one cell is deposited on the reaction site of the substrate.

The apparatus shown in FIG. 2 consists of a housing 1 in which a chamber 2 for a liquid jacket flow is located. Furthermore, the apparatus has a cannula 3 for the conduction of a cell suspension, i.e. a suspension of cells in liquid. The chamber 2 for the jacket flow tapers downwardly to a nozzle 4. At the head of the apparatus there is a piezoceramic 5 which can exert periodic pressure fluctuations on the nozzle 4.

Furthermore the apparatus includes a laser source (not shown) which generates a laser beam 6 beneath the nozzle 4. Deflection plates 7 are provided beneath the laser beam 6 to which electrical potential can be applied on the purpose of generating an electrical field between the deflection plates 7.

In order to carry out the method of the invention a cell suspension with a predetermined cell concentration, for example a suspension of cells in cell culture medium is supplied via the cannula 3 into the apparatus and guided via an outlet 8 into the chamber 2. Parallel to this a jacketing liquid is fed through the inlet 9 with a high pressure into the chamber 2 and flows through it. As a result of the pressure of the jacketing liquid the liquid jet emerging from the outlet 8 is hydrodynamically focused and guided to the nozzle 4.

Through the piezoceramic 5 a piezoelectric modulation is applied to the nozzle 4 by which the nozzle 4 is exposed to periodic pressure fluctuations. As a result of these pressure fluctuations, individual liquid drops 10 are separated from the liquid stream at the nozzle 4. Thereafter the drops 10 fall downwardly as a result of the gravity and pass the laser beam 6 through which any fluorescence-marked antibodies bound to the cell membrane or fluorescent dyes incorporated in the cells can be detected. Prior to passing or after passing the laser beam 6 the individual liquid drops 10 are selectively or differentially electrically charged by means of a corresponding apparatus (not shown). That is to say individual liquid drops 10 receive an electrical charge whereas other liquid drops 10 remain electrically neutral or individual liquid drops 10 receive a positive electric charge whereas the remaining liquid drops 10 receive a negative electric charge or the individual liquid drops 10 each receive a different quantity of electrical charge, with the quantity of electrical charge applied per liquid drop 10 for example being proportional to the intensity of the fluorescence per liquid drop 10 detected by the laser beam. Thereafter the individual liquid drops 10 are guided through an electric field generated by deflection plates 7 in which electrically charged liquid drops 10 are deflected. Beneath the deflection plates 7 there is a substrate 11 in the form of an object carrier which is so arranged that liquid drops 10 with a specific electric charge are deflected onto a reaction site 12 of this substrate 11.

In this connection the parameters during guidance of the liquid suspension through the nozzle 4, during the separation of the liquid drops 10 from the nozzle 4 and during the guidance of the liquid drops 10 through the electrical field are so set that the eukaryotic cell(s) impinging on the reaction site 12 of the substrate 11 no longer have surrounding liquid or at least largely have no extra-cellular liquid.

The substrate 11 shown in FIG. 3a is of rectangular shape and has a total of 48 reaction sites 12 which are distributed on 6 rows arranged beneath one another which each have 8 reaction sites 12.

As can be seen in FIG. 3b each reaction site 12 has an inner central circularly designed hydrophilic region 13. This inner hydrophilic region 13 is concentrically surrounded at the outside by a circular ring-shaped (inner) hydrophobic region 14 which is in turn concentrically surrounded at the outside by a circular ring-like (middle) hydrophilic region 15. Finally the (middle) hydrophilic region 15 is surrounded at the outer side by an (outer) hydrophobic region 16.

Through this design of the reaction sites 12 a situation is achieved in which, after the deposition of at least one eukaryotic cell thereon, liquid drops form, from the liquid adhering to the at least one eukaryotic cell or from the liquid added to the at least one eukaryotic cell after the deposition on the reaction site, which adhere comparatively firmly to the substrate so that the following enzymatic reaction can be directly carried out on the reaction sites without the eukaryotic cells having to be transferred into a closed reaction vessel or the like. In this way work-intensive and time-intensive transfer steps are on the one hand avoided. Furthermore, it is made possible for a plurality of samples to be prepared in parallel on the substrate 11 without the danger existing that the liquid drops which lie spatially closely alongside one another can mix with one another with even smallest vibrations or as a result of running of the liquid drops as a consequence of a drop volume which is too large.

### REFERENCE NUMERAL LIST

1 housing

2 chamber for jacket flow

3 cannula for cell suspension

4 nozzle

5 piezoceramic

6 laser beam

7 deflection plates

8 outlet of the channels

9 chamber inlet

10 liquid drops

11 substrate

12 reaction site

13 inner hydrophilic region

14 inner hydrophobic region

15 middle hydrophilic region

16 outer hydrophobic region

1-33. (canceled)

34. A method for carrying out an enzymatic reaction with a sample containing at least one eukaryotic cell including the following steps:
a) making available a starting material containing at least one eukaryotic cell,
b) removal of at least one eukaryotic cell from the starting material,
c) coloring the cell nucleus or the cell nuclei of the eukaryotic cell(s),
d) depositing at least one eukaryotic cell on a reaction site (12) of a solid substrate (11) in a liquid volume of less than 10 μl,
e) detecting whether a colored cell nucleus is present on a reaction site (12) of the substrate (11) and also
f) carrying out an enzymatic reaction with the at least one eukaryotic cell on the substrate (11), with the enzymatic reaction being one of a PCR, LCR or RCA, wherein the method step e) is carried out after the method steps c) and d).

35. A method in accordance with claim 34, wherein a substrate (11) is used having a plurality of reaction sites (12) and wherein between 1 and 5 eukaryotic cells are deposited per reaction site (12) of the substrate (11) in the method step d).

36. A method in accordance with claim 35, wherein between 1 and 3 eukaryotic cells are deposited per reaction site (12) of the substrate (11) in the method step d).

37. A method in accordance with claim 35, wherein 1 or 2 eukaryotic cells are deposited per reaction site (12) of the substrate (11) in the method step d).

38. A method in accordance with claim 34, wherein the method step c) is carried out before or after the method step d) and before or after the method step b).

39. A method in accordance with claim 34, wherein, prior to or during the deposition of the at least one eukaryotic cell on a reaction site (12) of the substrate (11) in accordance with method step d), the absolute number of the eukaryotic cell(s) deposited or to be deposited per reaction site (12) is determined.

40. A method in accordance with claim 39, wherein the quantification of the absolute number of the at least one eukaryotic cell takes place microscopically, preferably with an optical microscope or a fluorescence microscope.

41. A method in accordance with claim 34, wherein the at least one eukaryotic cell is deposited in the method step d) on the inner hydrophilic region (13) of a reaction site (12) of the substrate (11) surrounded by a hydrophobic region (14).

42. A method in accordance with claim 41, wherein the substrate (11) has from 2 to 1000 different reaction sites (12) each including a substantially circular inner hydrophilic region (13), with the inner hydrophilic regions (13) each being concentrically surrounded by a hydrophobic region (14) of substantially circular ring-shape which is surrounded at the outer side by a middle hydrophilic region (15) which is substantially of circular ring-shape and is in turn surrounded at the outer side by an outer hydrophobic region (16).

43. A method in accordance with claim 42, wherein the substrate (11) has from 12 to 256 different reaction sites (12).

44. A method in accordance with claim 42, wherein the substrate (11) has from 24 to 96 different reaction sites (12).

45. A method in accordance with claim 42, wherein the substrate (11) has 48 different reaction sites (12).

46. A method in accordance with claim 34, wherein the substrate (11) is one of an object carrier and a microtiter plate.

47. A method in accordance with claim 34, wherein a substrate (11) is used having a plurality of reaction sites (12) and wherein the at least one eukaryotic cell is deposited in the method step d) in a liquid volume of less 5 μl than on a reaction site (12) of the substrate (11).

48. A method in accordance with claim 47, wherein the at least one eukaryotic cell is deposited in the method step d) in a liquid volume of less than 2 μl on a reaction site (12) of the substrate (11).

49. A method in accordance with claim 47, wherein the at least one eukaryotic cell is deposited in the method step d) in a liquid volume of less than 1 μl on a reaction site (12) of the substrate (11).

50. A method in accordance with claim 34, wherein a substrate is used having a plurality of reaction sites (12), wherein at least one said eukaryotic cell is deposited in the method step d) onto each of said reaction sites (12), wherein a liquid suspension containing the eukaryotic cells is guided through a nozzle (4), wherein the liquid flow at the nozzle is separated into individual liquid drops (10) separate from one another, with the individual liquid drops (10) each containing a predetermined number of eukaryotic cells, wherein all or some liquid drops (10) are electrically charged after the separation from the nozzle (4) and the individual liquid drops (10) are guided by an electrical field, whereby one or more electrically charged liquid drops (10) are deflected onto one or more reaction sites (12) of the substrate (11), wherein enzyme is subsequently added to the eukaryotic cells and wherein an enzymatic reaction is started.

51. A method in accordance with claim 50, wherein a reaction buffer is subsequently added to the eukaryotic cells.

52. A method in accordance with claim 50, wherein genetically different cells are present in the liquid suspension, an individual cell or a plurality of cells are marked with one of a fluorescence marked antibody and a fluorescent dye, one of the liquid flow and the individual liquid drops (10) are guided through a laser beam through which the fluorescence of the individual cells is measured, the individual liquid drops (10) separated from the liquid flow are electrically charged in dependence on the fluorescence of the at least one cell contained therein with a specific electrical charge and the individual liquid drops (10) are guided through an electrical field so that the liquid drops (10) having a pre-selected electrical charge are deflected onto the substrate (11).

53. A method in accordance with claim 34, wherein a substrate (11) is used having a plurality of reaction sites (12) and the at least one eukaryotic cell is deposited in the method step d) by means of a flow cytometer onto at least one reaction site (12) of said substrate (11).

54. A method in accordance with claim 34, wherein a substrate (11) is used having a plurality of reaction sites (12) and wherein at least one of the depositing of the at least one eukaryotic cell on a reaction site of the substrate in accordance with step d) and the removal of the at least one eukaryotic cell from the starting material in accordance with the method step b) takes place by one of laser capture microdissection and laser pressure catapultation.

55. A method in accordance with claim 34, wherein one or more substrates (11) are used which each have 2 to 1000 different reaction sites (12) each including an inner hydrophilic region (13) and wherein the individual numbers of the eukaryotic cell(s) deposited in the method step d) per reaction site (12) is stored during or after the method step d) on a data carrier.

56. A method in accordance with claim 34, wherein 12 to 256 reaction sites (12) different reaction sites (12) are provided on the or each said substrate (11).

57. A method in accordance with claim 56, wherein 24 to 96 different reaction sites (12) are provided on the or each said substrate (11).

58. A method in accordance with claim 34, wherein 48 different reaction sites (12) are provided on the or each said substrate (11).

59. A substrate (11) having at least one reaction site (12) on which one or more eukaryotic cells are provided which are obtainable by a method for carrying out an enzymatic reaction with a sample containing at least one eukaryotic cell including the following steps:
a) making available a starting material containing at least one eukaryotic cell,
b) removal of at least one eukaryotic cell from the starting material,
c) coloring the cell nucleus or the cell nuclei of the eukaryotic cell(s),
d) depositing at least one eukaryotic cell on a reaction site (12) of a solid substrate (11) in a liquid volume of less than 10 μl,
e) detecting whether a colored cell nucleus is present on a reaction site (12) of the substrate (11) and also
f) carrying out an enzymatic reaction with the at least one eukaryotic cell on the substrate (11), with the enzymatic reaction being one of a PCR, LCR or RCA, wherein the method step e) is carried out after the method steps c) and d).
